(19) United States
(12) Patent Application Publication
(10) Pub. No.: US 2025/0082176 A1
(43) Pub. Date: Mar. 13, 2025

(54) MEDICAL IMAGING DEVICE AND MEDICAL OBSERVATION SYSTEM

(71) Applicants: Sony Olympus Medical Solutions Inc., Tokyo (JP); Sony Group Corporation, Tokyo (JP)

(72) Inventors: Satoshi TAIRA, Tokyo (JP); Yuuya TANAKA, Tokyo (JP)

(73) Assignees: Sony Olympus Medical Solutions Inc., Tokyo (JP); Sony Group Corporation, Tokyo (JP)

(21) Appl. No.: 18/824,947

(22) Filed: Sep. 5, 2024

(30) Foreign Application Priority Data
Sep. 12, 2023 (JP) 2023-147959

(51) Int. Cl. A61B 1/00

(52) U.S. Cl. CPC A61B 1/000095; A61B 1/00006; A61B 1/00013

(57) ABSTRACT

A medical imaging device includes: a plurality of image sensors each configured to capture a subject image to output a pixel signal; and a signal integration unit configured to convert a plurality of the pixel signals output from the plurality of image sensors into pixel signals corresponding to a specific transmission standard.

### CROSS-REFERENCE TO RELATED APPLICATION

This application claims priority from Japanese Application No. 2023-147959, filed on Sep. 12, 2023, the contents of which are incorporated by reference herein in its entirety.

### BACKGROUND

The present disclosure relates to a medical imaging device and a medical observation system.

In the related art, a medical observation system is known in which an observation target (subject such as a person) is irradiated with visible light such as white light or excitation light that is narrow band light emitted from a light source device to observe the observation target (See, for example, WO 2021/039869 A).

The medical observation system described in WO 2021/039869 A has a normal observation mode and a fluorescence observation mode. In the medical observation system, in the case of the normal observation mode, the observation target is irradiated with visible light such as white light, an image sensor captures return light (reflected light) of the visible light from the observation target, and a normal light image captured by the image sensor is displayed on a display device. On the other hand, in the medical observation system, in the case of the fluorescence observation mode, the observation target is irradiated with excitation light that is narrow band light, the image sensor captures fluorescence emitted from a substance contained in the observation target in response to the irradiation of the excitation light, and a fluorescence image captured by the image sensor is displayed on the display device.

### SUMMARY

In the medical observation system described in WO 2021/039869 A, only one image sensor captures the return light of the visible light and the fluorescence. In a case where only one image sensor is used as described above, it is necessary to use an image sensor sensitive to both visible light and fluorescence for imaging, and the sensitivity is not sufficient to capture weak fluorescence, which makes it difficult to generate a fluorescence image suitable for observation.

To address this, it is probable that the following two image sensors are provided according to the wavelength of light to be captured: an image sensor that captures return light of visible light and an image sensor that captures fluorescence.

However, in a case where such a configuration is adopted and circuits that separately process pixel signals from the two image sensors in compliance with a transmission standard corresponding to the image sensors are provided, a problem arises in which the circuit scale increases and the power consumption increases.

There is a need for a medical imaging device and a medical observation system that are capable of preventing an increase in a circuit scale and power consumption.

According to one aspect of the present disclosure, there is provide a medical imaging device including: a plurality of image sensors each configured to capture a subject image to output a pixel signal; and a signal integration unit configured to convert a plurality of the pixel signals output from the plurality of image sensors into pixel signals corresponding to a specific transmission standard.

According to another aspect of the present disclosure, there is provide a medical observation system including: a medical imaging device configured to capture a subject image to output a pixel signal; and a signal processing device configured to process the pixel signal output from the medical imaging device, wherein the medical imaging device includes a plurality of image sensors each configured to capture a subject image to output a pixel signal, and a signal integration unit configured to convert a plurality of the pixel signals output from the plurality of image sensors into pixel signals corresponding to a specific transmission standard.

### BRIEF DESCRIPTION OF THE DRAWINGS

FIG. 1 is a diagram illustrating a configuration of a medical observation system according to an embodiment;

FIG. 2 is a block diagram illustrating a configuration of a camera head and a control device;

FIG. 3 is a diagram illustrating functions of first and second signal receiving units;

FIG. 4 is a diagram illustrating buffer processing;

FIG. 5 is a diagram illustrating a first modification to the embodiment;

FIG. 6 is a diagram illustrating a second modification to the embodiment;

FIG. 7 is a diagram illustrating a third modification to the embodiment; and

FIG. 8 is a diagram illustrating a fourth modification to the embodiment.

### DETAILED DESCRIPTION

Hereinafter, modes for carrying out the present disclosure (hereinafter, referred to as embodiments) will be described with reference to the drawings. Note that the present disclosure is not limited to the embodiments described below. Further, in the description of the drawings, the same portions are denoted by the same reference signs.

### Schematic Configuration of Medical Observation System

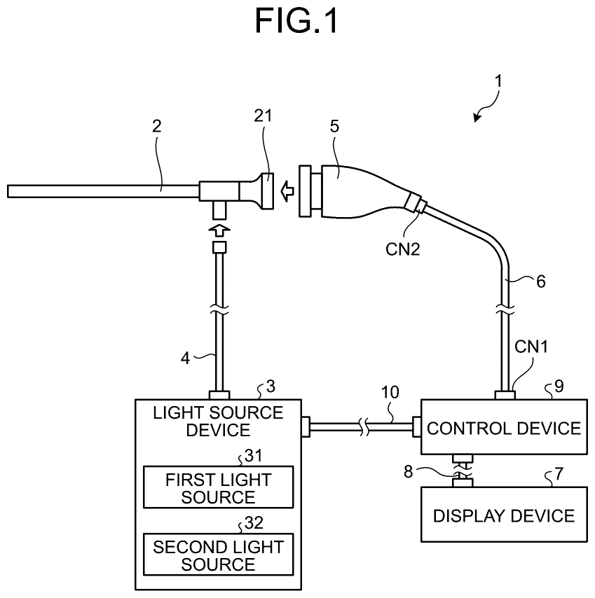

FIG. 1 is a diagram illustrating the configuration of a medical observation system 1 according to an embodiment.

The medical observation system 1 according to the present embodiment is a system that is used in the medical field to observe the inside of a living body. As illustrated in FIG. 1, the medical observation system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 is configured with a rigid endoscope. Specifically, the insertion unit 2 is entirely rigid or partially rigid with a partially flexible portion and has an elongated shape. The insertion unit 2 is inserted into a living body. The insertion unit 2 includes an optical system (not illustrated) having one or a plurality of lenses to condense light from a subject (subject image).

The light source device 3 is connected to one end of the light guide 4, and supplies light to be applied to inside of the living body to that one end of the light guide 4 under the control of the control device 9. As illustrated in FIG. 1, the light source device 3 includes a first light source 31 and a second light source 32.

The first light source 31 emits light having a first wavelength band. In the present embodiment, the first light source 31 is configured with a light emitting diode (LED) that emits white light (light having the first wavelength band (visible light)).

The second light source 32 emits excitation light having a second wavelength band different from the first wavelength band. In the present embodiment, the second light source 32 is configured with a semiconductor laser that emits near-infrared excitation light having a near-infrared wavelength band (light having the second wavelength band (narrow band light)). The excitation light is not limited to the near-infrared excitation light, and excitation light having other wavelength bands may be used.

The near-infrared excitation light emitted by the second light source 32 is excitation light that excites a fluorescent substance such as indocyanine green. When excited by the near-infrared excitation light, the fluorescent substance such as indocyanine green emits fluorescence with a central wavelength on a long wavelength side as compared with a central wavelength of the wavelength band of the near-infrared excitation light.

In the present embodiment, the light source device 3 is configured separately from the control device 9, but the present disclosure is not limited thereto. A configuration in which the light source device 3 is provided in the same housing as the control device 9 may be adopted.

The light guide 4 has one end detachably connected to the light source device 3 and the other end detachably connected to the insertion unit 2. The light guide 4 transmits light (white light or near-infrared excitation light) supplied from the light source device 3 from one end to the other end of the light guide 4 to supply the light to the insertion unit 2. The light supplied to the insertion unit 2 (white light or near-infrared excitation light) is emitted from the distal end of the insertion unit 2 and applied to the inside of the living body. In a case where the inside of the living body is irradiated with white light, the white light reflected in the living body is condensed by the optical system in the insertion unit 2. Hereinafter, for convenience of description, the white light condensed by the optical system in the insertion unit 2 is referred to as a first subject image. In a case where the inside of the living body is irradiated with near-infrared excitation light, the near-infrared excitation light reflected in the living body and fluorescence emitted from the excited fluorescent substance, such as indocyanine green accumulated at a lesion in the living body, are condensed by the optical system in the insertion unit 2. Hereinafter, for convenience of description, the near-infrared excitation light and the fluorescence condensed by the optical system in the insertion unit 2 are referred to as a second subject image.

The camera head 5 corresponds to a medical imaging device according to the present disclosure. The camera head 5 is detachably connected to a proximal end of the insertion unit 2 (eyepiece unit 21 (FIG. 1)). The camera head 5 captures, under the control of the control device 9, the first subject image (white light) or the second subject image (near-infrared excitation light and fluorescence) condensed by the insertion unit 2 to generate a pixel signal (hereinafter, referred to as a captured image).

Note that a detailed configuration of the camera head 5 will be described in “Configuration of Camera Head” described later.

The first transmission cable 6 has one end detachably connected to the control device 9 via a connector CN1 (FIG. 1) and the other end detachably connected to the camera head 5 via a connector CN2 (FIG. 1). The first transmission cable 6 transmits a captured image and the like output from the camera head 5 to the control device 9, and transmits a control signal, a synchronization signal, a clock, power, and the like output from the control device 9 to the camera head 5.

Note that, in the transmission of a captured image and the like from the camera head 5 to the control device 9 via the first transmission cable 6, the captured image and the like may be transmitted as an optical signal or may be transmitted as an electric signal. The same applies to the transmission of a control signal, a synchronization signal, and a clock from the control device 9 to the camera head 5 via the first transmission cable 6.

The display device 7 is configured with a display using liquid crystal, organic electro luminescence (EL), or the like, and displays an image based on a video signal from the control device 9 under the control of the control device 9.

The second transmission cable 8 has one end detachably connected to the display device 7 and the other end detachably connected to the control device 9. The second transmission cable 8 transmits the video signal processed by the control device 9 to the display device 7.

The control device 9 corresponds to a signal processing device according to the present disclosure. The control device 9 is configured with a central processing unit (CPU), a field-programmable gate array (FPGA), and the like, and has control over the operations of the light source device 3, the camera head 5, and the display device 7.

Note that a detailed configuration of the control device 9 will be described in “Configuration of Control device” described later.

The third transmission cable 10 has one end detachably connected to the light source device 3 and the other end detachably connected to the control device 9. The third transmission cable 10 transmits a control signal from the control device 9 to the light source device 3.

### Configuration of Camera Head

Next, the configuration of the camera head 5 will be described.

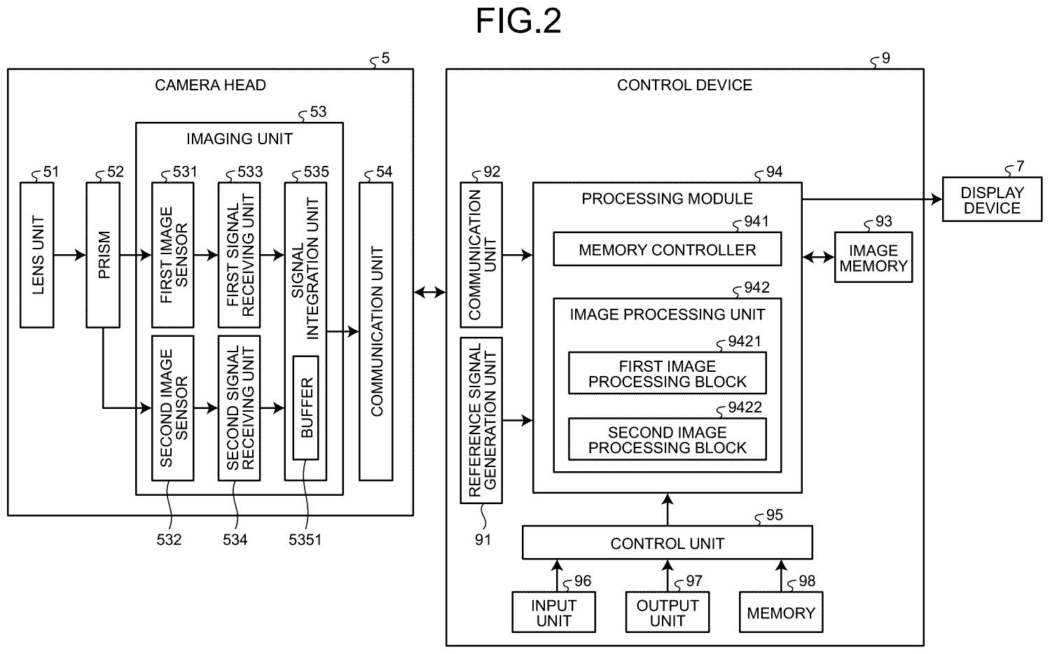

FIG. 2 is a block diagram illustrating the configuration of the camera head 5 and the control device 9.

As illustrated in FIG. 2, the camera head 5 includes a lens unit 51, a prism 52, an imaging unit 53, and a communication unit 54.

The lens unit 51 includes one or a plurality of lenses. The lens unit 51 forms the first subject image (white light) condensed by the insertion unit 2 on an imaging surface of a first image sensor 531 (FIG. 2), and forms the second subject image (near-infrared excitation light and fluorescence) condensed by the insertion unit 2 on an imaging surface of a second image sensor 532 (FIG. 2).

The prism 52 separates the first subject image (white light) and the second subject image (near-infrared excitation light and fluorescence) through the lens unit 51. The prism 52 causes the first subject image (white light) to travel toward the first image sensor 531. The prism 52 causes the second subject image (near-infrared excitation light and fluorescence) to travel toward the second image sensor 532.

The imaging unit 53 captures an image of the inside of the living body under the control of the control device 9. As illustrated in FIG. 2, the imaging unit 53 includes the first image sensor 531, the second image sensor 532, a first signal receiving unit 533, a second signal receiving unit 534, and a signal integration unit 535.

The first and second image sensors 531 and 532 are each configured with a charge coupled device (CCD), a complementary metal oxide semiconductor (CMOS), or the like that receives incident light to convert the light into an electric signal. Note that the number of image sensors according to the present disclosure is not limited to two of the first and second image sensors 531 and 532, and three or more image sensors may be used.

Here, although not specifically illustrated, the first image sensor 531 includes an invalid region that is not electrically guaranteed, an optical black region (OB region), and a valid pixel region in which to convert the first subject image formed by the lens unit 51 into an imaging signal to output the imaging signal. Similarly, the second image sensor 532 includes an invalid region, an optical black region (OB region), and a valid pixel region.

The first image sensor 531 captures the first subject image (white light) via the prism 52 under the control of the control device 9. Hereinafter, for convenience of description, a captured image generated by capturing the first subject image (white light) by the first image sensor 531 is referred to as a normal light image. In the present embodiment, the first image sensor 531 has a number of pixels capable of capturing an image at a resolution of 4K. That is, the normal light image is a 4K image having the number of pixels of 4K. The number of pixels of the first image sensor 531 is not limited to 4K, and the first image sensor 531 may be an image sensor having a number of pixels capable of capturing an image at other resolutions.

The second image sensor 532 captures the second subject image (near-infrared excitation light and fluorescence) via the prism 52 under the control of the control device 9. Hereinafter, for convenience of description, a captured image generated by capturing the second subject image (near-infrared excitation light and fluorescence) by the second image sensor 532 is referred to as a fluorescence image. In the present embodiment, the second image sensor 532 has a number of pixels capable of capturing an image at a resolution of HD. That is, the fluorescence image is an HD image having the number of pixels of HD. The number of pixels of the second image sensor 532 is not limited to HD, and the second image sensor 532 may be an image sensor having a number of pixels capable of capturing an image at other resolutions. Further, an excitation light cut filter that removes only at least a part of the near-infrared excitation light traveling toward the second image sensor 532 may be disposed on a preceding stage side of the optical path of the second image sensor 532.

As described above, the camera head 5 includes the first and second image sensors 531 and 532 that output different types of images (normal light image (4K image) and fluorescence image (HD image)), respectively.

The first signal receiving unit 533 reads a pixel signal (normal light image) from each pixel in the first image sensor 531 based on a signal related to an imaging timing and cycle in the first image sensor 531. Here, examples of the signal related to the imaging timing and cycle in the first image sensor 531 include a clock and a synchronization signal (vertical/horizontal synchronization signal).

The second signal receiving unit 534 reads a pixel signal (fluorescence image) from each pixel in the second image sensor 532 based on a signal related to an imaging timing and cycle in the second image sensor 532. Here, examples of the signal related to the imaging timing and cycle in the second image sensor 532 include a clock and a synchronization signal (vertical/horizontal synchronization signal).

Note that detailed functions of the first and second signal receiving units 533 and 534 will be described later in “Functions of First and Second Signal Receiving Units”.

The signal integration unit 535 performs, on pixel signals (normal light image and fluorescence image) read by the first and second signal receiving units 533 and 534 respectively, signal processing such as processing of removing reset noise, processing of multiplying an analog gain for amplifying the corresponding pixel signal (analog signal), A/D conversion, integration processing and buffer processing described below under the control of the control device 9.

Here, the integration processing is processing of converting the pixel signals (normal light image and fluorescence image) read by the first and second signal receiving units 533 and 534 respectively into pixel signals corresponding to a specific transmission standard. In the present embodiment, the specific transmission standard is a transmission standard corresponding to an image sensor having the fastest transmission rate or an image sensor having the largest amount of signal data of the pixel signal among the first and second image sensors 531 and 532. That is, the specific transmission standard is a 4K transmission standard. The transmission standard includes a size of a captured image in the vertical and horizontal directions, a transmission time required to transmit one frame, a horizontal and vertical synchronization timing, and a reference clock. Therefore, in the integration processing according to the present embodiment, a fluorescence image signal of an HD transmission standard is converted into a pixel signal of the 4K transmission standard. Note that the specific transmission standard according to the present disclosure is not limited to the 4K transmission standard, and other transmission standards may be adopted.

The buffer processing is processing using a buffer 5351 (FIG. 2) provided in the signal integration unit 535.

Details of the buffer processing will be described later in “Buffer Processing”.

The communication unit 54 functions as a transmitter that transmits captured images sequentially output from the imaging unit 53 to the control device 9 via the first transmission cable 6. The communication unit 54 is configured, for example, with a high-speed serial interface that communicates a captured image with the control device 9 via the first transmission cable 6 at a transmission rate of 1 Gbps or higher. In the present embodiment, the communication unit 54 alternately transmits the normal light image and the fluorescence image that have been subjected to the signal processing including the integration processing in the signal integration unit 535 to the control device 9 in a time division manner.

Note that, in the present embodiment, the configuration in which the imaging unit 53 of the medical imaging device includes two image sensors of the first image sensor and the second image sensor has been described in detail, but the present disclosure is not limited thereto. The imaging unit may be a stereoscopic observation device in which an imaging unit has a left eye and a right eye, for example, a stereoscopic endoscope device. In the stereoscopic observation device, the left eye may include two or more image sensors having different transmission standards, and the right eye may also include two or more image sensors having different transmission standards. Further, in the stereoscopic observation device, the signal integration unit 535 may be provided in each of the left eye and the right eye, or one signal integration unit 535 may be provided for both the left eye and the right eye.

### Configuration of Control Device

Next, the configuration of the control device 9 will be described with reference to FIG. 2.

As illustrated in FIG. 2, the control device 9 includes a reference signal generation unit 91, a communication unit 92, an image memory 93, a processing module 94, a control unit 95, an input unit 96, an output unit 97, and a storage unit (memory) 98.

The reference signal generation unit 91 generates a signal serving as the reference necessary for the operations of the camera head 5 and the control device 9.

The reference signal is a clock and a synchronization signal (vertical/horizontal synchronization signal). In the present embodiment, the reference signal generation unit 91 generates different clocks and synchronization signals as a clock and a synchronization signal to be output to the first and second image sensors 531 and 532.

The communication unit 92 functions as a receiver that receives captured images sequentially output from the camera head 5 (communication unit 54) via the first transmission cable 6. The communication unit 92 is configured, for example, with a high-speed serial interface that communicates a captured image with the communication unit 54 at a transmission rate of 1 Gbps or higher.

The image memory 93 is configured, for example, with a dynamic random access memory (DRAM) or the like. The image memory 93 may temporarily store a plurality of frames of captured images sequentially output from the camera head 5.

The processing module 94 processes the captured images sequentially received via the communication unit 92. As illustrated in FIG. 2, the processing module 94 includes a memory controller 941 and an image processing unit 942.

The memory controller 941 controls writing of a captured image into the image memory 93 and reading of the captured image from the image memory 93. More specifically, the memory controller 941 writes a normal light image received by the communication unit 92 into the image memory 93, reads the normal light image from the image memory 93 at a specific timing, and inputs the normal light image to a first image processing block 9421 in the image processing unit 942. Further, the memory controller 941 writes a fluorescence image received by the communication unit 92 into the image memory 93, reads the fluorescence image from the image memory 93 at a specific timing, and inputs the fluorescence image to a second image processing block 9422 in the image processing unit 942.

The image processing unit 942 performs image processing on the captured images that are sequentially received via the communication unit 92 and read from the image memory 93 by the memory controller 941. In addition, the image processing unit 942 generates a display image (video signal for display) for displaying the captured image that has been subjected to the image processing. The image processing unit 942 then outputs the display image to the display device 7. As a result, the display image is displayed on the display device 7.

As illustrated in FIG. 2, the image processing unit 942 includes the first image processing block 9421 that performs image processing on an input normal light image and the second image processing block 9422 that performs image processing on an input fluorescence image.

Here, examples of the image processing performed by the first and second image processing blocks 9421 and 9422 include, as the first image processing, optical black subtraction processing, white balance adjustment processing, demosaic processing, color correction matrix processing, gamma correction processing, YC processing of converting RGB signals into luminance and chrominance signals (Y, Cb/Cr signals), gain adjustment, noise removal, and filter processing for structure enhancement.

Note that the image processing performed by the first image processing block 9421 and the image processing performed by the second image processing block 9422 may be different from each other, or may be the same image processing.

The control unit 95 is implemented by executing various programs stored in the storage unit 98 by a controller such as a CPU or a micro processing unit (MPU), and the control unit 95 controls the operations of the light source device 3, the camera head 5, and the display device 7 and also controls the entire operation of the control device 9. The control unit 95 is not limited to the CPU or the MPU, and may be configured with an integrated circuit such as an application specific integrated circuit (ASIC) or an FPGA.

The input unit 96 is configured using an operating device such as a mouse, a keyboard, and a touch panel, and receives a user operation by a user such as a surgeon. The input unit 96 outputs an operation signal corresponding to the user operation to the control unit 95.

The output unit 97 is configured using a speaker, a printer, or the like, and outputs various types of information.

The storage unit 98 stores therein a program executed by the control unit 95, information necessary for processing of the control unit 95, and the like.

### Functions of First and Second Signal Receiving Units

Next, the functions of the first and second signal receiving units 533 and 534 will be described.

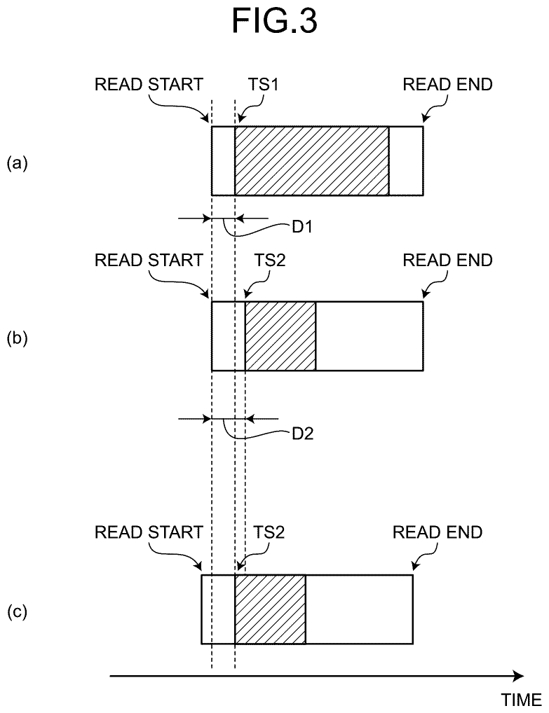

FIG. 3 is a diagram illustrating the functions of the first and second signal receiving units 533 and 534.

Specifically, (a) of FIG. 3 is a diagram illustrating the function of the first signal receiving unit 533 that reads a pixel signal (normal light image) having the number of pixels of 4K. Further, (b) of FIG. 3 and (c) of FIG. 3 are diagrams illustrating the function of the second signal receiving unit 534 that reads a pixel signal (fluorescence image) having the number of pixels of HD. In FIG. 3, each of the normal light image and the fluorescence image to be read is represented by a rectangle. In (a) of FIG. 3, a hatched region in a rectangle representing the normal light image indicates a pixel signal from the valid pixel region of the first image sensor 531. In each of (b) of FIG. 3 and (c) of FIG. 3, a hatched region in a rectangle representing the fluorescence image indicates a pixel signal from the valid pixel region of the second image sensor 532.

The first signal receiving unit 533 reads a pixel signal (normal light image) from each pixel of the first image sensor 531 based on the clock and the synchronization signal (vertical/horizontal synchronization signal) generated by the reference signal generation unit 91. In (a) of FIG. 3, the reference sign “TS1” represents the leading edge timing of the pixel signal from the valid pixel region of the first image sensor 531. In the normal light image, the data amount of the pixel signal from the read start timing to the leading edge timing TS1 is a data amount D1.

The second signal receiving unit 534 reads a pixel signal (fluorescence image) from each pixel of the second image sensor 532 based on the clock and the synchronization signal (vertical/horizontal synchronization signal) generated by the reference signal generation unit 91. In (b) of FIG. 3 and (c) of FIG. 3, the reference sign “TS2” represents the leading edge timing of the pixel signal from the valid pixel region of the second image sensor 532. In the fluorescence image, the data amount of the pixel signal from the read start timing to the leading edge timing TS2 is a data amount D2.

Meanwhile, the data amount D2 is greater than the data amount D1. Therefore, in a case where the read start timings of both the normal light image and the fluorescence image are set to be the same timing, the leading edge timing TS1 and the leading edge timing TS2 are different from each other as illustrated in (a) of FIG. 3 and (b) of FIG. 3.

To cope with this, in the present embodiment, as illustrated in (c) of FIG. 3, the second signal receiving unit 534 advances the read start timing by a time corresponding to the difference between the data amounts D1 and D2 so that the leading edge timing TS1 and the leading edge timing TS2 occur at the same time.

Meanwhile, the leading edge timing TS1 and the leading edge timing TS2 may be made to occur at the same time by delaying the read start timing by the first signal receiving unit 533 by a time corresponding to the difference between the data amounts D1 and D2.

### Buffer Processing

Next, the buffer processing executed by the signal integration unit 535 will be described.

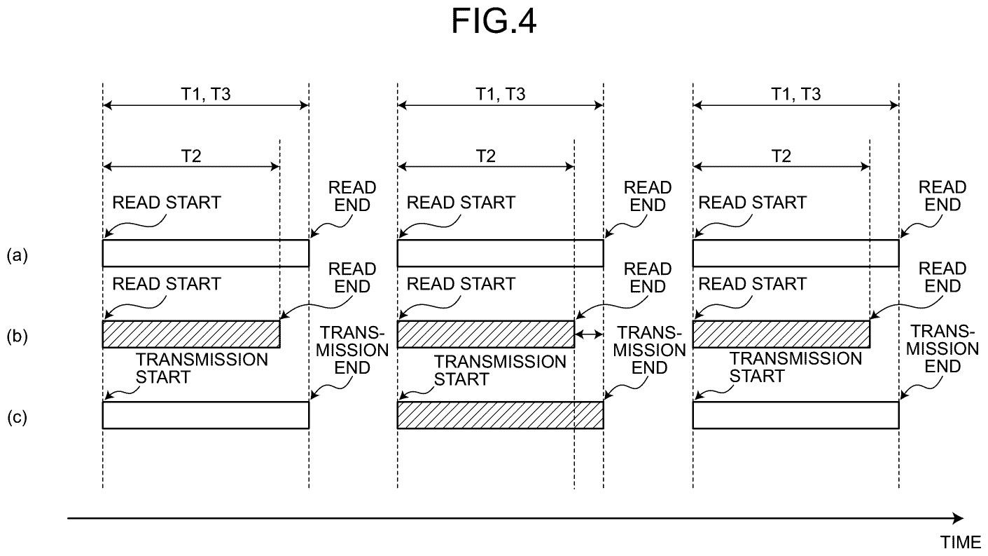

FIG. 4 is a diagram illustrating the buffer processing. Specifically, (a) of FIG. 4 is a diagram corresponding to (a) of FIG. 3. In (a) of FIG. 4, illustration of the pixel signal from the valid pixel region of the first image sensor 531 is omitted. Further, (b) of FIG. 4 is a diagram corresponding to (b) of FIG. 3. In (b) of FIG. 4, illustration of the pixel signal from the valid pixel region of the second image sensor 532 is omitted. Further, in order to distinguish between the normal light image and the fluorescence image, the normal light image is represented as a white rectangle, and the fluorescence image is represented as a hatched rectangle. Further, (c) of FIG. 4 illustrates the normal light image and the fluorescence image alternately transmitted from the communication unit 54 in a time division manner.

Meanwhile, in a case where a clock and a synchronization signal with recommended specifications for the first and second image sensors 531 and 532 are used as the clock and the synchronization signal output from the reference signal generation unit 91 to the first and second image sensors 531 and 532, that is, in a case where the clock and the synchronization signal input to the first and second image sensors 531 and 532 are not the same, as illustrated in (a) of FIG. 4 and (b) of FIG. 4, a read time T2 of the fluorescence image is shorter than a read time T1 of the normal light image. In other words, the read time T2 of the fluorescence image is shorter than each transmission time T3 of the normal light image and the fluorescence image.

Accordingly, in order to absorb the difference between the read time T2 and the transmission time T3, the signal integration unit 535 executes buffer processing of reading the pixel signal (fluorescence image) read from the second signal receiving unit 534 while temporarily storing that pixel signal into the buffer 5351. In other words, the signal integration unit 535 executes the buffer processing to absorb the difference between the clock and the synchronization signal input to the first and second image sensors 531 and 532. As a result, as illustrated in (c) of FIG. 4, the fluorescence image may also be transmitted at the transmission time T3 similarly to the normal light image.

According to the present embodiment described above, the following effects are obtained.

The camera head 5 according to the present embodiment includes the signal integration unit 535 that converts the pixel signals output from the first and second image sensors 531 and 532 into pixel signals corresponding to the 4K transmission standard.

This eliminates the need for circuits that separately process the pixel signals from the first and second image sensors 531 and 532 in compliance with a transmission standard corresponding to the image sensors. Therefore, according to the camera head 5 of the present embodiment, it is possible to prevent an increase in circuit scale and power consumption.

Further, the signal integration unit 535 executes the buffer processing to absorb the difference between the clock and the synchronization signal input to the first and second image sensors 531 and 532.

Therefore, in a case where the normal light image and the fluorescence image are processed on the control device 9 side, processing may be performed at a specified frequency, and no design changes are necessary on the control device 9 side.

The camera head 5 includes the first and second signal receiving units 533 and 534 that read pixel signals from the first and second image sensors 531 and 532, respectively. The first and second signal receiving units 533 and 534 read the pixel signals such that the leading edge timings TS1 and TS2 of the pixel signals from the valid pixel regions in the first and second image sensors 531 and 532 are the same timing.

Therefore, since the control device 9 receives data on the valid pixel region in each of the first and second image sensors 531 and 532 at a specified frequency, processing may be performed at the specified frequency, which eliminates the need to change the design on the control device 9 side.

### Other Embodiments

Although the embodiment for carrying out the present disclosure has been described so far, the present disclosure should not be limited only to the embodiment described above.

Configurations of first to fourth modifications described below may be adopted.

### First Modification

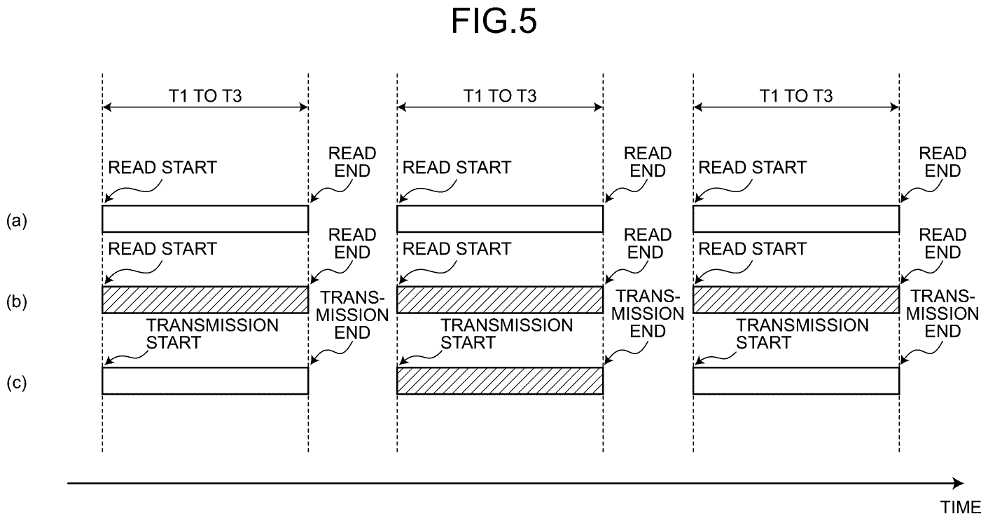

FIG. 5 is a diagram illustrating the first modification to the embodiment. Specifically, FIG. 5 is a diagram corresponding to FIG. 4.

In the embodiment described above, the reference signal generation unit 91 generates different clocks and synchronization signals as clocks and synchronization signals to be output to the first and second image sensors 531 and 532. However, the present disclosure is not limited thereto.

The reference signal generation unit 91 according to the first modification generates, as the clocks and the synchronization signals to be output to the first and second image sensors 531 and 532, the same clock and synchronization signal.

As a result, as illustrated in FIG. 5, the read time T2 of the fluorescence image becomes the same as the read time T1 of the normal light image. In other words, the read time T2 of the fluorescence image becomes the same as each transmission time T3 of the normal light image and the fluorescence image.

According to the first modification described above, the following effect is achieved in addition to the same effects as those of the above-described embodiment.

According to the first modification, the buffer processing described in the above-described embodiment is unnecessary, which eliminates the need for the buffer 5351.

### Second Modification

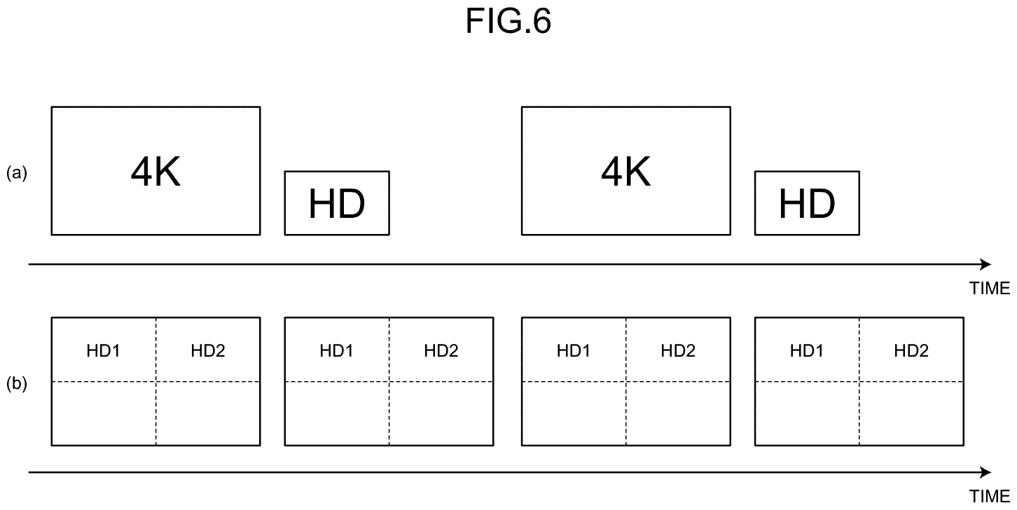

FIG. 6 is a diagram illustrating the second modification to the embodiment. Specifically, FIG. 6 schematically illustrates an image to be transmitted to the control device 9 by the communication unit 54 with the horizontal axis representing time. In FIG. 6, the letter “4K” is added to an image having the number of pixels of 4K, and the letter “HD”, “HD1”, or “HD2” is added to an image having the number of pixels of HD.

In the embodiment described above, for example, first and second modes to either of which the control unit 95 switches according to a user operation on the input unit 96 may be adopted.

Similarly to the embodiment, the first mode is a mode in which a fluorescent substance such as indocyanine green is used and fluorescence from the fluorescent substance is travelled toward the second image sensor 532 side by the prism 52. Similarly to the embodiment, in the first mode, a normal light image and a fluorescence image simultaneously captured by the first and second image sensors 531 and 532 are converted by the signal integration unit 535 in compliance with the 4K transmission standard, and then are alternately transmitted in a time division manner toward the control device 9 by the communication unit 54 ((a) of FIG. 6).

The second mode is a mode in which a fluorescent substance different from that in the embodiment is used. In the second mode, fluorescence from the fluorescent substance is travelled toward the first image sensor 531 side by the prism 52 similarly to white light. That is, the wavelength band of the fluorescence overlaps with the wavelength band of the white light. In the second mode, among first and second periods alternately repeated, the control unit 95 turns on the first light source 31 in the first period and turns on the second light source 32 in the second period. Further, the first image sensor 531 captures an image for each of the first and second periods that are alternately repeated, in synchronization with the lighting timings of the first and second light sources 31 and 32. As a result, the imaging unit 53 captures an image in the first period, and generates a normal light image having the number of pixels of HD by thinning reading or pixel accumulation. The imaging unit 53 also captures an image in the second period, and generates a fluorescence image having the number of pixels of HD by thinning reading or pixel accumulation. Further, the imaging unit 53 (signal integration unit 535) converts each of the normal light image having the number of pixels of HD and the fluorescence image having the number of pixels of HD into the 4K transmission standard and outputs the resultant image. Then, the communication unit 54 simultaneously transmits the normal light image (HD image) and the fluorescence image (HD image) generated by the imaging unit 53 ((b) of FIG. 6). In (b) of FIG. 6, the letter “HD1” is added to the normal light image (HD image), and the letter “HD2” is added to the fluorescence image (HD image).

According to the second modification described above, the following effects are achieved in addition to the same effects as those of the above-described embodiment.

According to the second modification, it is possible to select the first or second mode according to the type of the fluorescent substance, and improve convenience.

### Third Modification

The medical observation system according to the third modification is a medical observation system using a so-called video scope (flexible endoscope) having an imaging unit on the distal end side of the insertion unit. Hereinafter, for convenience of description, the medical observation system 1 according to the third modification is referred to as a medical observation system 1A.

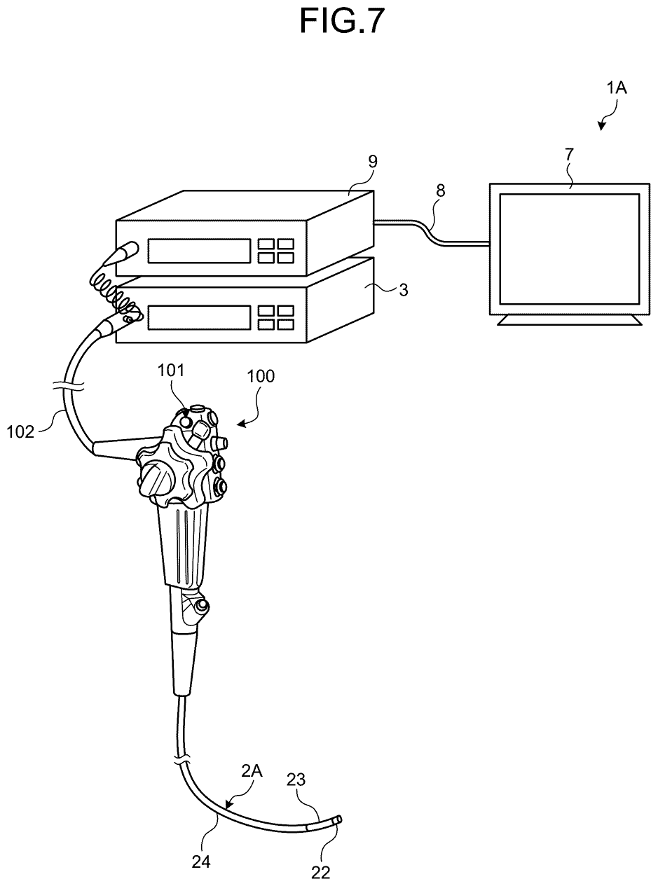

FIG. 7 is a diagram illustrating the third modification to the embodiment.

As illustrated in FIG. 7, the medical observation system 1A includes an endoscope 100 that captures an in-vivo image of an observed region by inserting an insertion unit 2A into a living body to output the captured image, the light source device 3 that generates illumination light emitted from a distal end of the endoscope 100, the control device 9 that processes the captured image output from the endoscope 100, and the display device 7 that is connected to the control device 9 via the second transmission cable 8 and displays an image based on a video signal processed by the control device 9.

As illustrated in FIG. 7, the endoscope 100 includes the insertion unit 2A having an elongated shape that is flexible, an operating unit 101 connected to a proximal end side of the insertion unit 2A and receiving various operations, and a universal cord 102 extending in a direction different from a direction in which the insertion unit 2A extends from the operating unit 101 and incorporating various cables connected to the light source device 3 and the control device 9.

As illustrated in FIG. 7, the insertion unit 2A includes a distal end unit 22, a bendable curve portion 23 connected to the proximal end side of the distal end unit 22 and configured with a plurality of curve pieces, and an elongated flexible tube portion 24 connected to the proximal end side of the curve portion 23 and having flexibility.

Although not specifically illustrated, the distal end unit 22 incorporates substantially the same configuration as that of the camera head 5 described in the embodiment. The captured image captured by the distal end unit 22 (imaging unit) is output to the control device 9 via the operating unit 101 and the universal cord 102.

Even in a case where the configuration of the third modification described above is adopted, the same effects as those of the embodiment described above are achieved.

### Fourth Modification

A medical observation system according to the fourth modification is a medical observation system using a surgical microscope that enlarges and captures a predetermined field of view inside a subject (inside a living body) or on a surface of the subject (surface of the living body). Hereinafter, for convenience of description, the medical observation system 1 according to the fourth modification is referred to as a medical observation system 1B.

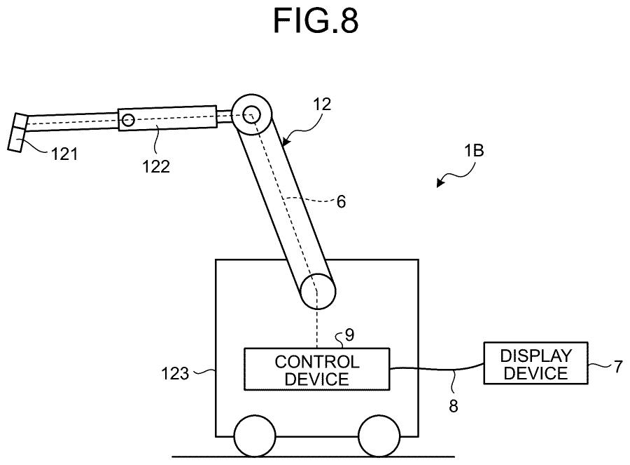

FIG. 8 is a diagram illustrating the fourth modification to the embodiment.

As illustrated in FIG. 8, the medical observation system 1B includes a surgical microscope 12 that captures an image used for observing a subject to output the captured image, the control device 9 that processes the captured image output from the surgical microscope 12, and the display device 7 that is connected to the control device 9 via the second transmission cable 8 and displays an image based on a video signal processed by the control device 9.

As illustrated in FIG. 8, the surgical microscope 12 includes a microscope unit 121 that enlarges and captures a micro portion of a subject to output the captured image, a support unit 122 that is connected to a proximal end portion of the microscope unit 121 and includes an arm that rotatably supports the microscope unit 121, and a base unit 123 that rotatably holds a proximal end portion of the support unit 122 and is movable on a floor surface.

As illustrated in FIG. 8, the control device 9 is disposed in the base unit 123. Although not specifically illustrated, the light source device 3 that generates illumination light to be emitted from the surgical microscope 12 to the subject is also provided in the base unit 123.

The base unit 123 may be fixed to a ceiling, a wall surface, or the like to support the support unit 122, instead of being movably provided on the floor surface.

Although not specifically illustrated, substantially the same configuration as the camera head 5 described in the embodiment is incorporated in the microscope unit 121. The captured image captured by the microscope unit 121 (imaging unit) is output to the control device 9 via the first transmission cable 6 wired along the support unit 122.

Even in a case where the configuration of the fourth modification described above is adopted, the same effects as those of the embodiment described above are achieved.

Note that the following configurations also belong to the technical scope of the present disclosure.

According to the medical imaging device and the medical observation system of the present disclosure, it is possible to prevent the increase in circuit scale and power consumption.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical imaging device comprising:
a plurality of image sensors each configured to capture a subject image to output a pixel signal; and
a signal integration unit configured to convert a plurality of the pixel signals output from the plurality of image sensors into pixel signals corresponding to a specific transmission standard.

2. The medical imaging device according to claim 1, wherein the specific transmission standard is a transmission standard that corresponds, among the plurality of image sensors, to an image sensor having a fastest transmission rate or an image sensor having a largest amount of signal data of the pixel signal.

3. The medical imaging device according to claim 1, further comprising a communication unit configured to transmit, in a time division manner, the plurality of pixel signals output from the plurality of image sensors and converted into the specific transmission standard by the signal integration unit.

4. The medical imaging device according to claim 1, wherein signals related to an imaging timing and cycle in the plurality of image sensors are set to be a same.

5. The medical imaging device according to claim 1, wherein
the signal integration unit includes a buffer configured to temporarily store at least one of the plurality of pixel signals, and
in a case where signals related to an imaging timing and cycle in the plurality of image sensors are not a same, the buffer is configured to store at least one of the plurality of pixel signals to absorb a difference between the signals related to the imaging timing and cycle in the plurality of image sensors.

6. The medical imaging device according to claim 1, further comprising a plurality of signal receiving units each configured to read respective one of the plurality of pixel signals from respective one of the plurality of image sensors, wherein
each of the plurality of signal receiving units is configured to read the respective one of the plurality of pixel signals such that leading edge timings of the pixel signals from valid pixel regions in the plurality of image sensors are a same timing.

7. The medical imaging device according to claim 1, further comprising a communication unit configured to be switchable between a first mode and a second mode, wherein
in the first mode, the communication unit is configured to transmit, in a time division manner, the plurality of pixel signals simultaneously captured by the plurality of image sensors and converted into the specific transmission standard by the signal integration unit, and
in the second mode, the communication unit is configured to simultaneously transmit the plurality of pixel signals captured in a time division manner by any one of the plurality of image sensors in compliance with the specific transmission standard.

8. The medical imaging device according to claim 1, wherein the plurality of image sensors includes:
a first image sensor configured to output a pixel signal of a 4K image; and
a second image sensor configured to output a pixel signal of an HD image.

9. A medical observation system comprising:
a medical imaging device configured to capture a subject image to output a pixel signal; and
a signal processing device configured to process the pixel signal output from the medical imaging device, wherein
the medical imaging device includes
a plurality of image sensors each configured to capture a subject image to output a pixel signal, and
a signal integration unit configured to convert a plurality of the pixel signals output from the plurality of image sensors into pixel signals corresponding to a specific transmission standard.
